(19) United States
(12) Patent Application Publication
(10) Pub. No.: US 2025/0037606 A1
(43) Pub. Date: Jan. 30, 2025

(54) AUTOMATICALLY GENERATING PERSONALIZED PATHWAYS TO GREATER HAPPINESS, POSITIVISM, MINDFULNESS AND WELLBEING

(71) Applicant: Balakrishna Menon, Reston, VA (US)

(72) Inventor: Balakrishna Menon, Reston, VA (US)

(21) Appl. No.: 18/916,252

(22) Filed: Oct. 15, 2024

Related U.S. Application Data
(63) Continuation of application No. 18/316,578, filed on May 12, 2023, now abandoned.
(60) Provisional application No. 63/341,371, filed on May 12, 2022.

(51) Int. Cl. G09B 19/00; G16H 20/70

(52) U.S. Cl. CPC G09B 19/00; G16H 20/70

(57) ABSTRACT

A facility creates automatic pathways for individuals to achieve greater happiness, positivism, mindfulness, wellbeing, and good health. System, data structures and processes, exchanges and transactions are described for enabling individuals and groups of connected people to achieve greater happiness, positivism, mindfulness, and wellbeing in their lives through this facility. The facility draws on emerging research from a number of fields, including but not limited to, psychology, psychophysiology, neurosciences, and behavioral sciences, and uses large language models, machine learning models, neural network models, and other computational and technological methods, tools, and devices that allows individuals to draw up their own unique pathway(s) to greater happiness, positivism, mindfulness, wellbeing, and good health, and employs personalized and unique nudges, actions, and rewards to motivate each individual pursue his or her own personalized pathway(s).

### CROSS-REFERENCE TO RELATED APPLICATIONS

This application is a continuation of U.S. application Ser. No. 18/316,578, filed May 12, 2023, and entitled “Automatically Generating Personalized Pathways to Greater Fun, Happiness, Mindfulness and Wellbeing,” which claims the benefit of provisional U.S. Application No. 63/341,371, filed May 12, 2022, and entitled “Automatically Generating Personalized Pathways to Greater Fun, Happiness, Mindfulness and Wellbeing,” which are hereby incorporated by reference in their entireties.

### BACKGROUND

Individuals are seen as seeking to enhance their personal mindfulness and wellbeing through actions and activities that generate happiness and positivism in them.

### BRIEF DESCRIPTION OF THE DRAWINGS

FIG. 1 provides a simplified and stylized breakdown of factors determining happiness of people.

FIG. 2 provides a simplified and stylized taxonomy of happiness.

FIG. 3 illustrates interactions between pleasure enhancing, personality developing and relationship building activities in producing happiness.

FIG. 4 is a flowchart showing a process performed by the facility in some embodiments in order to increase happiness.

FIG. 5 is a dataflow diagram showing a process performed by the facility in some embodiments in order to increase happiness.

FIG. 6 depicts the flowchart for the initial set up and integration of the different blocks and elements of the facility.

FIG. 7 depicts the flowchart for the systematic operation and integration of the different blocks and elements of the facility.

FIG. 8 present an elaboration of the model to individual fun, happiness, mindfulness, and wellbeing.

FIG. 9 elaborates Happiness Nudges and Happiness Actions with illustrative examples.

FIG. 10 shows the components of the facility that offers a number of services to the individual/user.

FIG. 11a shows development of the initial Happiness Nudge Model based on directly provided and/or indirectly sourced individual/user information.

FIG. 11b shows the generation of Happiness Nudges and Actions through individual/user request, and the engagement of partner entities, where necessary, to cause Happiness Actions.

FIG. 12a shows Happiness Assessments generated, in some embodiments, directly through individual/user requests.

FIG. 12b shows Happiness Assessments generated, in some embodiments, indirectly through Happiness Nudges and Happiness Actions of the individual/user.

FIG. 12c shows how, in some embodiments, Happiness Assessment and Happiness Nudges and Actions feed back into the Happiness Nudge Model to improve its predictive power.

FIG. 13 shows Happiness Tracker generated, in some embodiments, through user request based on information from Happiness Nudges and Actions and Happiness Assessments of the individual/user, at any point or over a period of time.

FIG. 14 shows Happiness Journal generated, in some embodiments, directly through individual/user request for journaling.

FIG. 15 shows Happiness Maps generated, in some embodiments, directly through individual/user request for Maps, based on information from Happiness Nudges and Actions and Happiness Assessments of the individual/user, tracked through Happiness Tracker or otherwise, at any point or over a period of time.

FIG. 16 shows Happiness Organizer generated, in some embodiments, through individual/user requests, for organizing behaviors, activities and way of life based on supportive Happiness Nudges and Actions for greater fun, happiness, mindfulness and wellbeing over time.

FIG. 17 shows the Happiness Ecosystem of individual/user comprising, in some embodiments, Happiness Actions, Happiness Assessments, Happiness Tracker and Maps, Happiness Journal, Happiness Organizer and other services offered through the facility.

FIG. 18a shows input of initial data/information, including initial registration and set up data, either directly by the individual/user, or indirectly through choices made or avoided by the individual/user, using one or more modalities of inputting the data/information.

FIG. 18b shows input of request by the individual/user and operation of the facility, in some embodiments, for Happiness Nudges and Actions and other components of the facility that offers a number of services to the individual/user, including, but not exclusively, Happiness Assessments, and Happiness Journal and Maps, Happiness Organizer, and Happiness Ecosystem.

FIG. 19 is a high-level block diagram of typical elements in which a software, hardware, and/or firmware facility implementing the functions of the facility operates in some embodiments.

FIG. 20 is a block diagram showing some of the components typically incorporated in at least some of the computer systems and other devices on which the facility operates.

### DETAILED DESCRIPTION

The inventor has recognized that happiness is not accidental. It needs to be cultivated and channeled carefully, and there is a science behind it which enables individuals optimize their happiness and positivism in their everyday lives, thereby enhance their mindfulness and wellbeing.

The inventor hypothesizes that, with the aid of technology, happiness levels of an individual/user at a point of time or over a period of time can be reliably predicted, based on a number of variables, chief among them: (i) their character traits (among others, the “Big Five”-openness, extraversion, conscientiousness, agreeableness, and neuroticism) which have genetic basis, (ii) life circumstances which are determined by demographic, socioeconomic, and environmental factors, (iii) endowments and resources available to the individual/user, (iv) other psychophysiological factors, and (v) individual/user's own actions in their daily lives. The inventor also hypothesizes that, based on the prediction of an individual/user's level of happiness, it is possible for the individual/user to be stimulated with personalized and context sensitive happiness actions through a combination of cues, prompts and rewards and technology-aided modalities to support the realization of preferred happiness action that enables the individual/user to chart out unique pathways towards greater personal happiness, positivism, mindfulness, and wellbeing. Each individual/user is also able to ascertain progress and make dynamic changes to his/her course, over time.

The facility (1) generates predictions about individual's/user's level of happiness at a given point and/or over a period of time, (2) generates personalized and context specific behavioral nudges that provides positive choices to the individual/user to make suitable alterations to their lives, which lead them to higher levels of happiness, and (3) provides a supporting partner platform that enables the individual/user execute necessary actions for realizing the nudges and thus enhancing their level of happiness or state of emotional wellbeing, over time.

The facility performs the following in some embodiments:
Develop individual/user profiles based on individual/user data obtained through direct individual/user inputs and indirect/inferred methods.
Analyzes data about individual/user's personality/character traits, life circumstances, endowments and resources, past levels of happiness, and relevant psychophysiological factors and draws inferences.
Predict individual/user's level of happiness/state of emotional wellbeing at or over a given period of time with a Happiness Predictor Model.
Draws on data related to location, environmental factors, context, resources, and endowments to generate person-specific and context-sensitive behavioral cues, actions, and pathways (Happiness Nudges and Actions) for greater happiness, positivism, mindfulness, and wellbeing, and provides Happiness Assessments, Happiness Organizer, and Happiness Journal, and other features.
Enables individuals/users to improve their immediate and longer-term happiness and wellbeing.

Hereafter, happiness is used synonymously with personal fun, positivism, mindfulness, wellbeing and similar emotions and state of psychological, functional, and physiological welfare.

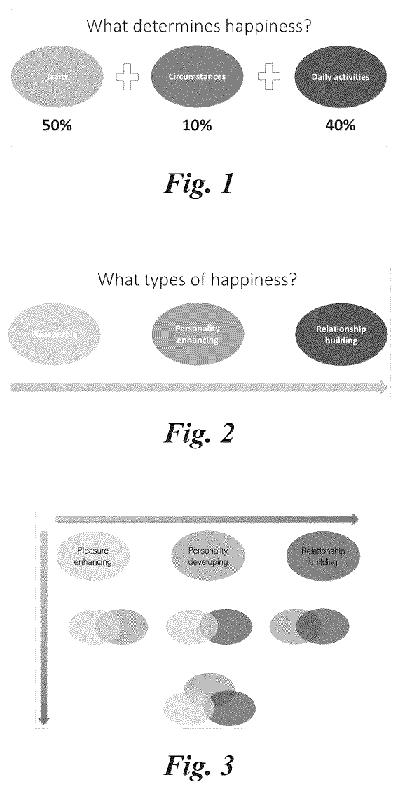

FIG. 1 provides a simplified and stylized breakdown of factors determining happiness of people. They comprise (1) genetic and other immutable and mutable factors, which are broadly called personality or character traits (contributing ˜50%), (2) personal, environmental, socioeconomic, and physical conditions which are broadly called circumstances (contributing˜10%), and (3) choices that individuals make, do not make, or avoid in their daily life which are broadly called daily activities (contributing˜40%).

FIG. 2 provides a simplified and stylized taxonomy of happiness, comprising of three broad typologies of actions and activities that generate fun, happiness, mindfulness, and wellbeing to individuals: (1) pleasurable activities, which are activities that may bring transient emotions of fun and/or happiness in everyday lives, (2) personality enhancing activities, which are activities that enhance positive emotions about the present and the future, such as zest, optimism, resilience, and accountability, and (3) relationship building activities, which are activities that promote values self-esteem, kindness, humor, empathy, forgiveness, and sharing.

In FIG. 3, the inventor postulates that happiness actions and activities moving rightward, from Pleasure enhancing to Relationship building, and moving downward, from happiness actions and activities that are either Pleasurable, Personality Enhancing or Relationship Enhancing to happiness actions and activities that are combinations of more than one typology bestow the individual/user with higher levels and/or more sustainable and long-term happiness, positivism, mindfulness, and wellbeing.

The inventor postulates that the level of happiness=f {trait characteristics, circumstances, resources and endowments, happiness levels in the immediately preceding time period, psychophysiological factors}.

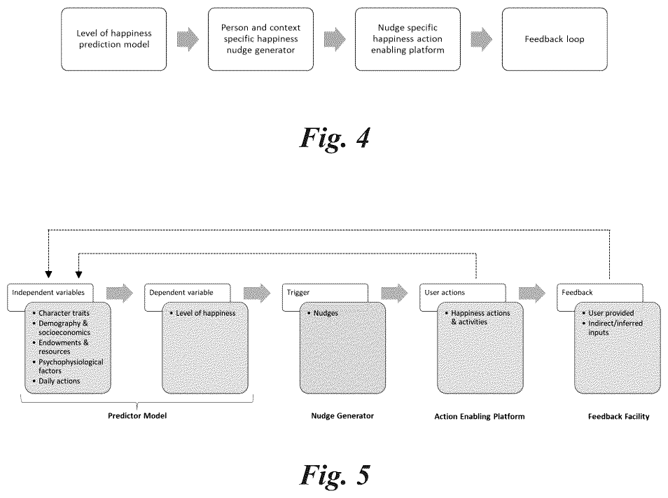

Based on the above, FIG. 4 and FIG. 5 together present the general setup of the facility, which includes (1) Happiness Predictor Model for predicting levels of happiness or state of emotional wellbeing of an individual/user at or over a period of time; (2) Happiness Nudge Generator for generating person-specific and context-sensitive behavioral nudges that enable the individual/user to be aware of and choose behavioral actions which lead to higher levels of happiness in the short-term, and to create pathways which lead to higher levels of positivity, mindfulness, and wellbeing in the long-term; (3) Happiness Action Enabling Platform for enabling the individual/user to execute happiness nudges; and (4) individual/user generated direct or indirect Feedback Facility.

Independent variables of the Model include (1) personality and character traits of the individual/user, (2) relevant demography and socioeconomic attributes, together termed as circumstances, of the individual/user, (3) endowments and resources associated with or available to the individual/user, (4) psychophysiological factors of the individual/user, inter alia heart rate variability, neurotransmitters, brain activity, cortisol levels, facial expressions, and micro-emotions, and (5) daily activities and habits of the individual/user that enable positive affect (e.g., social connections, mindfulness activities, physical exercise) or negative affect (e.g., lack of adequate sleep, unhealthy eating habits) on happiness, resulting in specific happiness levels at a point or over a period of time. Dependent variable or output of the model is the level of happiness state of emotional wellbeing of the individual/user at or over a period of time.

Based on the predicted level of happiness of the individual/user at or over a period of time, the Happiness Nudge Generator generates happiness nudges that prompts the individual/user towards actions which can lead to greater positivism, happiness, mindfulness, and wellbeing in their daily and long-term lives. User actions (affirm, negate, or ignore) are those performed by the individual/user based on the outputs of the model and the Nudge Generator. These User actions provide further inputs and insights to retrain the Model for improved predictability of happiness levels and nudges. Feedback derived directly or indirectly from individual/user provides inputs and insights to retrain the model.

The Happiness Predictor Model, the Happiness Nudge Generator, and the Happiness Action Platform of the facility set up based on the following steps.

### Stage 1:

1) Survey questionnaires are developed for control group.

2) Baseline survey is conducted of control group.

3) Happiness Predictor Model is set up and trained using control group data.

4) Happiness Predictor Model is tested for prediction accuracy.

5) Happiness Predictor Model is retrained and set up to predict individual/user level of happiness.

### Stage 2:

1) Adjusted survey questionnaires are developed for treatment group(s) in target market(s).

2) Survey is conducted of targeted treatment group(s).

3) Happiness Predictor Model is retrained with treatment group data.

4) Happiness nudges and actions are developed for each target market(s) based on survey information of treatment group(s).

5) Happiness Nudge Generator and Happiness Action Platform are developed.

6) Happiness Predictor Model, Happiness Nudge Generator, and Happiness Action Platform are validated in treatment group(s) in target market(s).

7) Happiness Predictor Model, Happiness Nudge Generator, and Happiness Action Platform are readied for deployment in target market(s).

Happiness survey involves the following steps:

1. Specific aspects of happiness for assessment are determined and happiness survey instrument is developed based on standard/widely recognized happiness survey scales (e.g., Positive and Negative Affect Schedule, Subjective Happiness Scale, or Satisfaction with Life Scale, etc.) and validated.

2. Population group to be surveyed is identified.

3. Appropriate sample size is computed and appropriate sampling strategy for the population being surveyed is determined. The sample size provides a representative sample of the population, while the sampling method is randomized and stratified.

4. Happiness survey instrument is administered to the selected sample using online and offline methods with options for participants provide unbiased and anonymous responses.

5. Survey data is cleaned up and analyzed using descriptive statistics, regression analyses and other statistical/machine learning methods.

6. Survey results are interpreted and draw conclusions about the variables that predict happiness in the population being surveyed.

7. Survey results are used to identify, classify, and group appropriate happiness actions with positive affect on individuals/users of target market(s) population.

Set up of the Happiness Predictor Model of the facility is as follows:

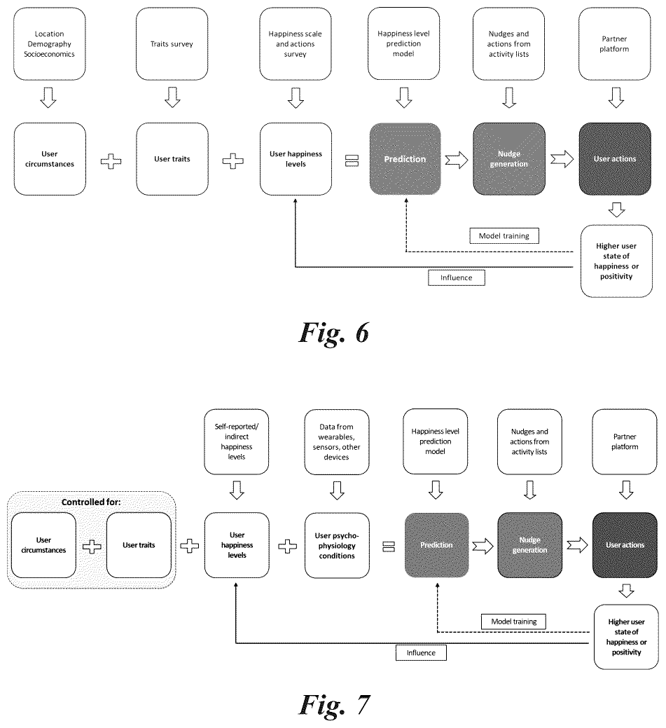

FIG. 6 depicts the flowchart for the initial set up and integration of the different blocks and elements of the facility. Independent variables that serve as input variables to set up and train the model are user circumstances (data gathered through surveys), user traits (data gathered through surveys on “Big Five” traits), and user happiness levels in the preceding day and preceding week (data obtained through surveys of happiness levels using modified versions of commonly used instruments such as Subjective Happiness Scale, Satisfaction with Life Scale, and/or Positive and Negative Affect Schedule). The outputs of the model are the predicted happiness level of an individual/user, context-sensitive and person-specific happiness nudges, and supporting actions. Person-specific and context-sensitive happiness nudges and actions are informed by additional data variables such as individual/user's location, available endowments, and resources, etc. Individual/user actions are executed through the partner platform.

FIG. 7 depicts the flowchart for the systematic operation and integration of the different blocks and elements of the facility. During systematic and ongoing operation of the facility, the Happiness Prediction Model is retrained iteratively using data on individual/user's happiness levels for the preceding hour/day/week, individual/user's psychophysiological factors, where accessible and reliable from wearable devices, sensors, and/or other devices or information sources. The inventor postulates that systematic and iterative retraining of the Happiness Prediction Model and Nudge Generator leads to improved prediction and nudges.

The inventor makes the claim that the unique Happiness Predictor Model has wider applications, inter alia, in predicting and/or analyzing individual/user emotional states of being, longer term personality and character traits, aptitudes, emotional regulation capabilities, productivity, susceptibilities to mental health issues, etc., and the invention may be put into applications related to these domains.

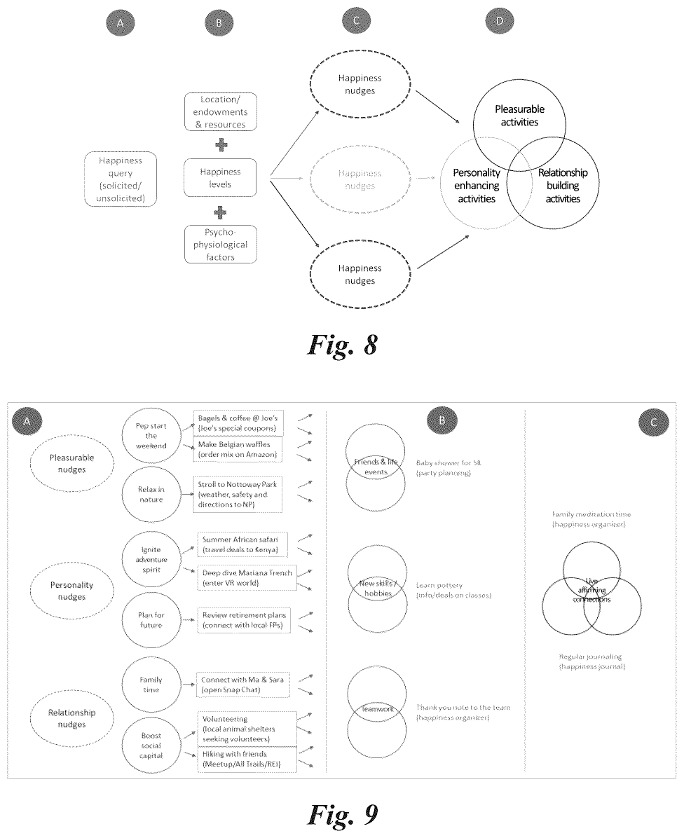

FIG. 8 presents an elaboration of how Pleasurable, Personality Enhancing, and Relationship Building nudges are generated through the happiness predictor and nudge generation model. (A) represents a happiness query triggered directly by the individual/user or indirectly by past individual/user actions or behavior patterns. (B) denotes inputs that support the happiness predictor and nudge generation model which generates the nudges. This includes inter alia, but is not limited to, information on (1) demographic and socioeconomic factors, location, resources and/or endowments available, and other specific attributes that may be unique to the individual/user at or over a given time period; (2) individual's personality traits, preferences, and other mutable and immutable characteristics, (3) information input directly or indirectly by the individual/user about his/her state of mood/feelings/emotion/temperament at or over a given time period; and 4) individual/user's psychophysiological factors that have a bearing on his/her happiness/state of emotional wellbeing. (A) and (B) constitute inputs for the happiness predictor and nudge generation model to generate (C) and (D), which are personalized Happiness Nudges for the individual/user that are Pleasurable, Personality Enhancing or Relationship Enhancing, or their combinations thereof. Happiness Nudges are behavior cues or stimulators that enable individuals/users to be aware of and choose actions and modify behaviors which make a positive difference to their happiness, positivism, mindfulness, and wellbeing ecosystem.

FIG. 9 elaborates Happiness Nudges and Happiness Actions with illustrative examples. Personalized Happiness Nudges lead to personalized Happiness Actions that are either Pleasurable, Personality Enhancing or Relationship Enhancing (A), or their combinations thereof (B) and (C). Based on individual/user responses to direct/indirect/inferred Happiness Assessments, Happiness Nudges and Actions, and iterative data from other input variables (happiness levels for preceding periods, other psychophysiological factors), the Happiness Prediction Model and the Nudge Generator are retrained and nudges and actions are generated, which provide reinforced and personalized pathways that enable individuals/users to achieve greater happiness, positivism, mindfulness, and wellbeing in their daily and longer-term lives.

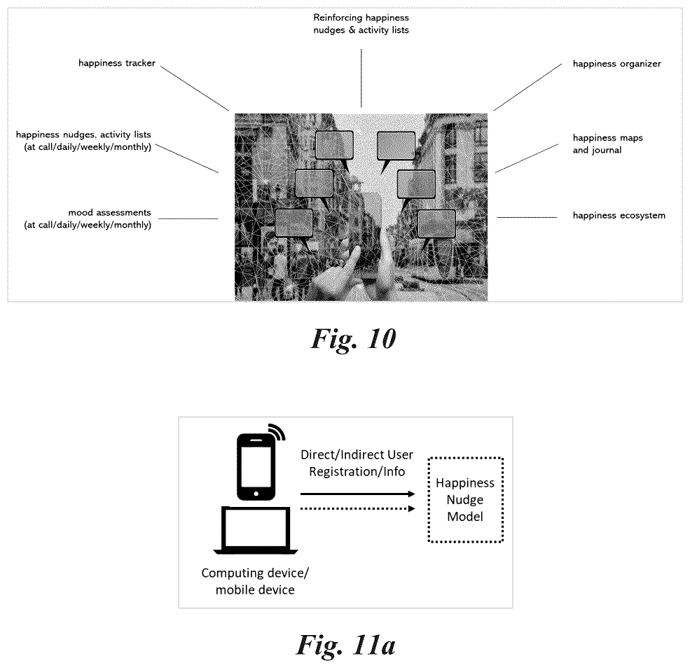

FIG. 10 shows the components of the facility that offers a number of services to the individual/user. This includes: (1) personalized Happiness Nudge and Activity Lists, (2) Happiness Assessments, based on scientific methods, (3) Happiness Tracker, drawing on Happiness Assessments, Happiness Nudges and Activity Lists of the individual/user, to enable the individual/user track and assess his/her happiness, mindfulness, and wellbeing over time, (4) Happiness Journal and Maps for the individual/user to map and/or record his/her experiences, feelings and insights, share and exchange with others as he/she desires, and chronicle pathways, including nudges, actions, settings, locations, people, and resources, to enhancing happiness, mindfulness and wellbeing, (5) Happiness Organizer to organize behaviors, activities and way of life for greater happiness, positivism, mindfulness, and wellbeing, and (6) Happiness Ecosystem, which brings together all of the happiness resources-cues, nudges, activities, relationships, and transactions—in a single place for the individual/user.

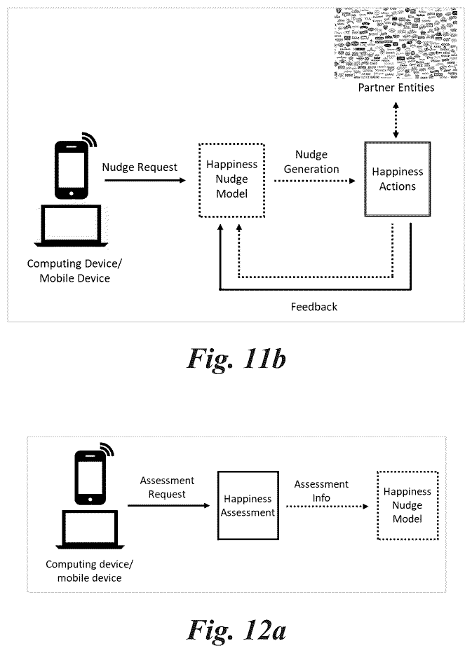

FIGS. 11 to 17 describe flow diagrams and system components that, in some embodiments, constitute the facility and its operations. In the FIGS. 9 to 17, bold lines indicate user-initiated flows and dashed lines indicate system-initiated flows, and bold boxes indicate frontend user-interface actions and boxes with dashed lines indicate backend system actions.

The facility for Automatically Generating Personalized Pathways to Greater Happiness, Positivism, Mindfulness and Wellbeing is pivoted on the Happiness Predictor and Nudge Generation Model which can predict the likely level of happiness/state of emotional wellbeing of the individual/user and generate Happiness Nudges and Actions that are contest-sensitive and person-specific. The other components associated with the facility operate by drawing on, feeding into and/or complementing the Happiness Predictor and Nudge Model. FIGS. 11a/b show Happiness Nudges and Actions generated by the individual/user in some embodiments. FIG. 11a shows initial triggering of the Happiness Predictor and Nudge Model based on directly provided and/or indirectly sourced individual/user information. FIG. 11b shows the generation of Happiness Nudges and Actions through individual/user request, and the engagement of partner entities through the Partner Platform, where necessary, to execute Happiness Actions.

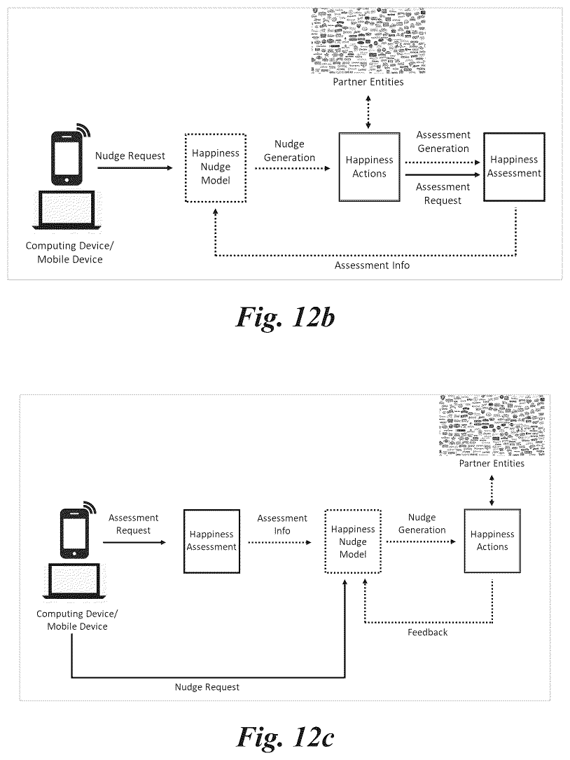

FIG. 12a/b/c show Happiness Assessments generated, in some embodiments, either directly through individual/user requests, or indirectly through Happiness Nudges and Happiness Actions of the individual/user. Happiness Assessment information iteratively trains the Happiness Predictor and Nudge Model to enhance the robustness of its predictive power. FIG. 12a/b/c show how, in some embodiments, Happiness Assessment and Happiness Nudges and Actions iteratively train the Happiness Predictor and Nudge Model to enhance the robustness its predictive power.

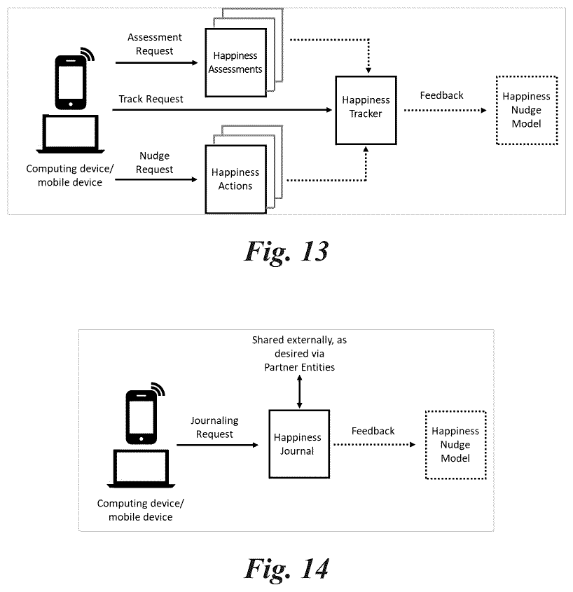

FIG. 13 shows Happiness Tracker generated, in some embodiments, through individual/user request based on information from Happiness Nudges and Actions and Happiness Assessments of the individual/user, at any point or over a period of time. Happiness Tracker trains the Happiness Predictor and Nudge Model, to improve its predictive power.

FIG. 14 shows Happiness Journal generated, in some embodiments, directly through individual/user request for journaling. Happiness Journal can be directly shared or exchanged by the individual/user with others using/following protocols, as desired, via Partner Entities (social media, chat, messaging, email, other media/communication facilities, etc.). Happiness Journal trains the Happiness Predictor and Nudge Model to enhance its predictive response.

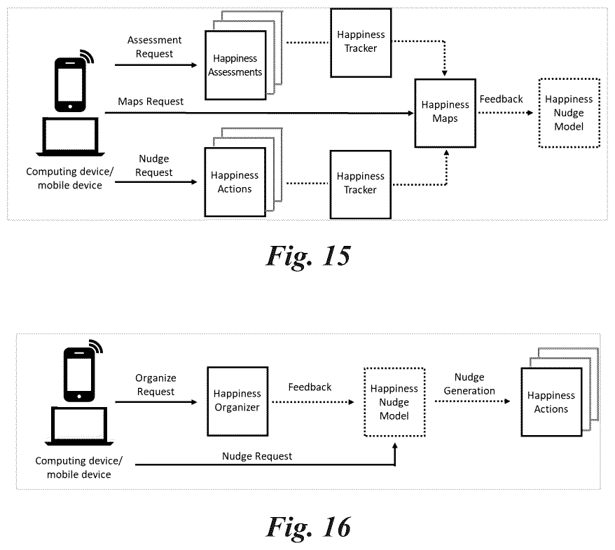

FIG. 15 shows Happiness Maps generated, in some embodiments, directly through individual/user request for Maps, based on information from Happiness Nudges and Actions and Happiness Assessments of the individual/user, tracked through Happiness Tracker or otherwise, at any point or over a period of time. Happiness Maps information feeds back into the Happiness Predictor and Nudge Model to enhance its predictive power.

FIG. 16 shows Happiness Organizer generated, in some embodiments, through individual/user requests, for organizing behaviors, activities and way of life based on supportive Happiness Nudges and Actions for greater fun, happiness, mindfulness and wellbeing over time. Happiness Organizer information feeds back into the Happiness Predictor and Nudge Model to enhance its predictive power.

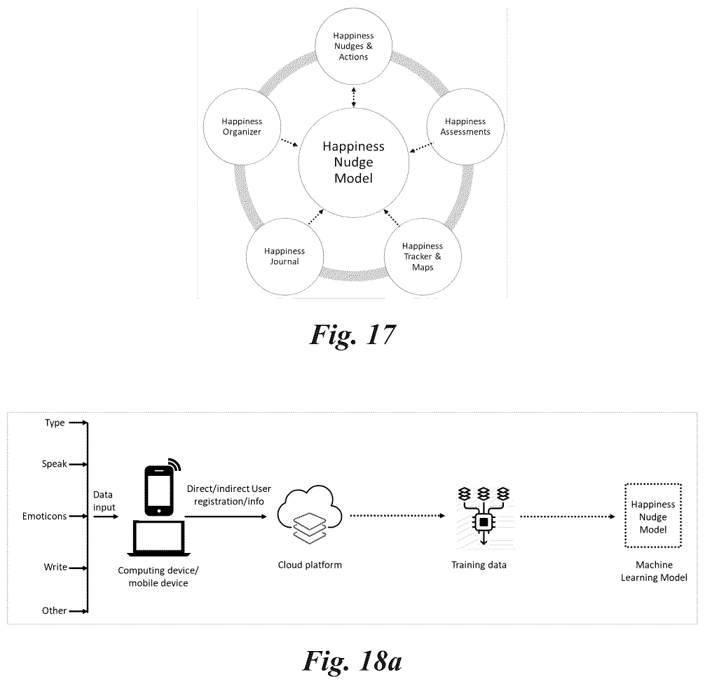

FIG. 17 shows the Happiness Ecosystem of individual/user comprising, in some embodiments, Happiness Nudges and Actions, Happiness Assessments, Happiness Tracker and Maps, Happiness Journal, Happiness Organizer and other services offered through the facility. The Happiness Ecosystem feeds into and is supported by the Happiness Predictor and Nudge Model.

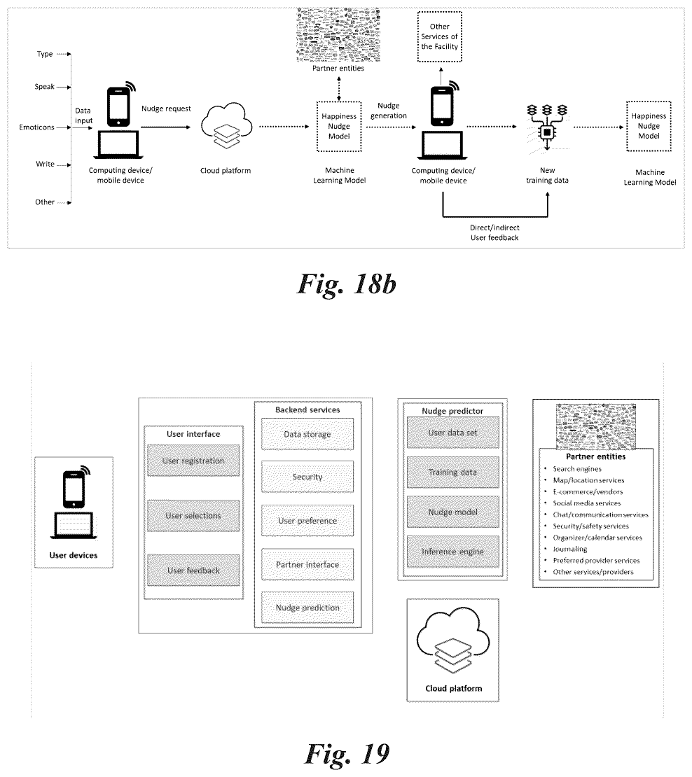

FIG. 18a/b show flow diagrams of key steps typically performed by the facility, in some embodiments, in order to set up the user-specific Happiness Predictor and Nudge Model for generating personalized and user-specific Happiness Nudges and Actions, Happiness Assessments, and Happiness Journal and Maps, Happiness Organizer, and Happiness Ecosystem. FIG. 18a shows input of initial data/information, including initial registration and set up data, either directly by the individual/user, or indirectly through choices made or avoided by the individual/user, using one or more modalities of inputting the data/information. This data/information informs the initial training data that is used to develop the machine learning model (Happiness Predictor and Nudge Model), from which the model creates and refines its rules. FIG. 18b shows input of request by the individual/user and operation of the facility, in some embodiments, for Happiness Nudges and Actions and other components of the facility that offers a number of services to the individual/user, including, but not exclusively, Happiness Assessments, and Happiness Journal and Maps, Happiness Organizer, and Happiness Ecosystem. Each operation of the facility generating Happiness Nudges and Actions and other services to the individual/user informs the training data that enables further recalibration and improvement of the machine learning model (Happiness and Predictor Nudge Model).

FIG. 19 is a high-level block diagram of typical elements in which a software, hardware, and/or firmware facility implementing the functions of the facility operates in some embodiments. This includes: (1) individual/user devices, to include, not exclusively, mobile phones, computing devices, wearables, sensors, and/or other devices (including, inter alia, devices associated with/attached to automobiles and entertainment systems); (2) individual/user interface, to include, not exclusively, user registration information, user selections for generating Happiness Nudges and follow on Happiness Actions, and user feedback provided directly by the individual/user or indirectly through engagement of components of the facility that offers various services to the individual/user; (3) backend systems service, to include, not exclusively, storage or individual/user data including preferences, other data including partner data, security systems and operations, and the nudge prediction model; (4) elements of the machine learning model (the Happiness Predictor and Nudge Model) to include, not exclusively, individual/user datasets, training data, machine learning model, and inference engine; (5) cloud platform, comprising servers, storage, databases, networking, software, analytics, and intelligence in an internet-based data center, for storage, processing and retrieval of various data; and (6) partner entities to include external facilities and services offered by third party and supporting the facility, in some embodiments, including, but not exclusively, search engines, location services, e-commerce, information services, communication services, and social media services.

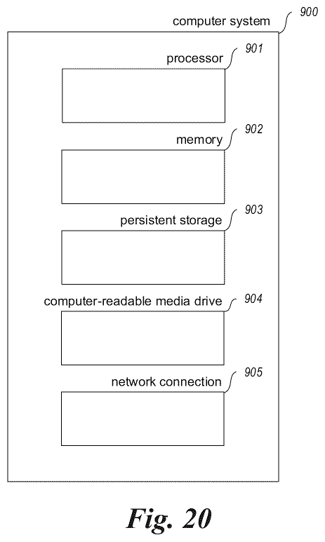

FIG. 20 is a block diagram showing some of the components typically incorporated in at least some of the computer systems and other devices on which the facility operates. In various embodiments, these computer systems and other devices 900 can include server computer systems, cloud computing platforms or virtual machines in other configurations, desktop computer systems, laptop computer systems, netbooks, mobile phones, personal digital assistants, televisions, cameras, automobile computers, electronic media players, physiological sensing devices, and/or their associated display devices, etc. In various embodiments, the computer systems and devices include zero or more of each of the following: a processor 901 for executing computer programs and/or training or applying artificial intelligence and machine learning models, such as a CPU, GPU, TPU, NNP, FPGA, or ASIC; a computer memory 902 for storing programs and data while they are being used, including the facility and associated data, an operating system including a kernel, and device drivers; a persistent storage device 903, such as a hard drive or flash drive for persistently storing programs and data; a computer-readable media drive 904, such as a floppy, CD-ROM, or DVD drive, for reading programs and data stored on a computer-readable medium; and a network connection 805 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations and having various components.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
determining a current set of characteristics of a person;
subjecting the determined characteristics to a sentiment prediction model to obtain a predicted level of happiness for the person; and
causing a nudge interaction to be made with the person that is based on the obtained predicted level of happiness.
